(19) United States
(12) Patent Application Publication
(10) Pub. No.: US 2018/0021380 A1
(43) Pub. Date: Jan. 25, 2018

(54) COMPOSITION INCLUDING MESENCHYMAL STEM CELL DERIVED FROM ADIPOSE TISSUES AND HYALURONIC ACID DERIVATIVE, METHOD OF PREPARING THE SAME, AND METHOD OF PREVENTING OR TREATING LOW BACK PAIN USING THE SAME

(71) Applicant: SUNGKWANG MEDICAL FOUNDATION, Seoul (KR)

(72) Inventors: Inbo HAN, Seongnam-si (KR); Soo-Hong LEE, Seongnam-si (KR)

(73) Assignee: SUNGKWANG MEDICAL FOUNDATION, Seoul (KR)

(21) Appl. No.: 15/655,273

(22) Filed: Jul. 20, 2017

(30) Foreign Application Priority Data
Jul. 21, 2016 (KR) 10-2016-0092903
Feb. 2, 2017 (KR) 10-2017-0015127

(51) Int. Cl. A61K 35/28; A61K 31/728

(52) U.S. Cl. CPC A61K 35/28; A61K 31/728

(57) ABSTRACT

Provided are a pharmaceutical composition for preventing or treating low back pain, the pharmaceutical composition including adipose tissue-derived mesenchymal stem cells and a hyaluronic acid derivative solution, and a method of preparing the same. Mesenchymal stem cells capable of differentiating into chondrocytes are mixed with a hyaluronic acid derivative solution prepared by using controlled reaction conditions of hyaluronic acid and hyaluronic acid derivative, for use as a composition for the prevention or treatment of diseases and low back pain caused by a change in degenerative change in intervertebral discs.

### CROSS-REFERENCE TO RELATED APPLICATIONS

This application claims the benefits of Korean Patent Application No. 10-2016-0092903, filed on Jul. 21, 2016, and 10-2017-0015127, filed on Feb. 2, 2017, in the Korean Intellectual Property Office, the disclosures of which are incorporated herein in their entirety by reference.

### BACKGROUND

### 1. Field

One or more embodiments relate to a pharmaceutical composition for preventing or treating low back pain, a method of producing the same and a method of preventing or treating low back pain using the same.

### 2. Description of the Related Art

Low back pain is a common disease that affects 84% of the population at least once in their lifetime. The main causes of low back pain are degenerative changes in the intervertebral disc, and degenerative changes in the intervertebral disc are irreversible diseases for which effective treatments do not exist. The intervertebral disc consists of nucleus pulosus and anulus fibrosus. The main components of the nucleus pulosus include a nucleus pulposus cell, proteoglycan, and type 2 collagen. The degenerative changes in the intervertebral disc are caused by a decrease in water content in the nucleus pulposus, the decrease resulting from a decrease in nucleus pulposus cell and a gradual decrease in proteoglycan and type 2 collagen. As a result, the posterior spinal joints and vertebrae may undergo general degenerative changes, resulting in severe pain and limitation in activity for the patient.

Conservative therapies such as medication and physical therapy are used to treat low back pain caused by the degenerative intervertebral disc. If conservative therapy is not effective, surgical treatments, such as intervertebral disc removal, spinal fusion, or artificial intervertebral disc insertion, may be considered. Surgical treatment has the effect of reducing pain for a short period of time, but in the long term, the degenerative changes may worsen and the spinal instability may cause more low back pain.

Adipose tissue-derived mesenchymal stem cells can be used as one of the cells capable of regenerating the intervertebral disc. Adipose tissue-derived mesenchymal stem cells have the ability to differentiate into osteoblasts, chondroblasts or adipocytes under appropriate differentiation conditions. There is a need for studies on biological and fundamental cell therapy for regenerating the intervertebral disc cartilage by using autogenous mesenchymal stem cells.

### SUMMARY

One or more embodiments include a pharmaceutical composition for preventing or treating low back pain, the pharmaceutical composition including adipose tissue-derived mesenchymal stem cells and a hyaluronic acid derivative solution.

One or more embodiments include a method of preparing a pharmaceutical composition for preventing or treating low back pain, the pharmaceutical composition including adipose tissue-derived mesenchymal stem cells and a hyaluronic acid derivative solution.

One or more embodiments include a method of preventing or treating low back pain using a pharmaceutical composition for preventing or treating low back pain, the pharmaceutical composition including adipose tissue-derived mesenchymal stem cells and a hyaluronic acid derivative solution.

Additional aspects will be set forth in part in the description which follows and, in part, will be apparent from the description, or may be learned by practice of the presented embodiments.

The inventors of the present application found that, when a hyaluronic acid derivative solution is administered to the intervertebral disc together with adipose tissue-derived mesenchymal stem cells, the hyaluronic acid derivative solution maintains a constant viscosity with respect to adipose tissue-derived mesenchymal stem cells and prevents the stem cells from leaking out, thereby more effectively treating the low back pain caused by the degenerative change in the intervertebral disc, and completed the present invention.

### BRIEF DESCRIPTION OF THE DRAWINGS

FIGS. 1A to 1C show results obtained by measuring the mechanical properties, degree of spread, viscosity, and viscosity using a rheometer of various concentrations of hyaluronic acid derivative solutions;

FIG. 2 is a flowchart to explain a method of preparing mesenchymal stem cells derived from adipose tissues for transplantation into the intervertebral disc;

FIG. 3 shows an image of the morphology of isolated mesenchymal stem cells according to a passage;

FIGS. 4A to 4C show graphs of the population doubling level (PDL), the doubling time (dT), and the cell viability rate according to a passage of cell groups of isolated mesenchymal stem cells;

FIG. 5 shows an image of mesenchymal stem cells differentiated into adipocytes, osteoblasts and chondrocytes after differentiation-inducing substances are added thereto;

FIG. 6 is a graph showing that surface factors of the isolated mesenchymal stem cells express mesenchymal stem cell markers;

FIGS. 7A and 7B are graphs showing the expression levels of CD44, CD73, CD90, CD105, CD45, CD29, CD49 and HLA-ABC in isolated mesenchymal stem cells;

FIG. 8 is a graph showing the expression level of TGF-β receptor III in isolated mesenchymal stem cells;

FIG. 9 shows an image of the morphology and karyotype of the separated mesenchymal stem cells after subculturing to the 7th passage;

FIG. 10 is a graph showing the Pfirrmann grade after administration of adipose derived mesenchymal stem cells and hyaluronic acid derivative solution in animals having induced intervertebral disc degeneration;

FIG. 11 shows images to explain experimental procedures performed according to Example 10;

FIG. 12 is a flowchart illustrating procedures of a clinical experiment for evaluating the pain of the intervertebral disc. Under the written consent of patients to participate in clinical trials, liposuction was performed in the subcutaneous fat layer of the patient's abdomen or thigh to obtain autologous adipose tissues therefrom. About three weeks after the liposuction, the autologous adipose tissue-derived mesenchymal stem cells and a hyaluronic acid derivative solution were injected, and the degree of intervertebral disc pain was evaluated after about 1 week, about 1 month, about 3 months, about 6 months, about 9 months, and about 12 months;

FIG. 13 shows MRI images of Patient 1 before and after transplantation of adipose tissue-derived mesenchymal stem cells and the hyaluronic acid derivative solution. Referring FIG. 13, Image a is of Patient 1 before the transplantation of adipose tissue-derived mesenchymal stem cell and hyaluronic acid derivative solution, Image b is of Patient 1 about 1 month after the transplantation, Image c is of Patient 1 about 6 months after the transplantation, and Image d is of Patient 1 about 12 months after the transplantation;

FIG. 14 is a graph showing visual analogue scale (VAS) scores before and after transplantation of adipose tissue-derived mesenchymal stem cell and hyaluronic acid derivative solutions in Patients 1 to 10; and

FIG. 15 is a graph showing oswestry disability index (ODI) scores before and after transplantation of adipose tissue-derived mesenchymal stem cell and hyaluronic acid derivative solutions in Patients 1 to 10.

### DETAILED DESCRIPTION

Hereinafter, embodiments of the present disclosure will be described in more detail. All technical terms used in connection with the present disclosure are used as having the same meanings as generally understood by one of ordinary skill in the art, unless otherwise defined. Also, although examples of methods or samples are described in this specification, similar or equivalent ones are also included in the scope of the present disclosure.

An aspect of the present disclosure provides a pharmaceutical composition for preventing or treating low back pain, the pharmaceutical composition including adipose tissue-derived mesenchymal stem cells and a hyaluronic acid derivative solution.

An aspect of the present disclosure provides a method of preventing or treating low back pain using a pharmaceutical composition for preventing or treating low back pain, the pharmaceutical composition including adipose tissue-derived mesenchymal stem cells and a hyaluronic acid derivative solution. The method may include administering to a subject in need thereof a therapeutically effective amount of the pharmaceutical composition. Stem cells refer to a cell capable of developing into any tissue. Stem cells have two basic features: self-renewal, that is, self-creation by repeated division; and multipotency, that is, differentiation into cells with specific functions depending on a particular environment. A mesenchymal stem cell (MSC) is a multipotential stem cell capable of differentiating into mesodermal cells such as bone, cartilage, fat, muscle cells, and ectodermal cells such as nerve cells. Adipose tissue-derived mesenchymal stem cells have a cell shape and immunophenotype that are similar to mesenchymal stem cells derived from bone marrow or umbilical cord blood, and have a higher yield because of the higher frequency of cell clustering than cord blood-derived mesenchymal stem cells. In addition, since adipose tissue is extracted from autologous fat, there is no danger of immunity rejection, and there is no ethical problem. Adipose tissue extraction or suction procedure is considered to cause relatively less pain and burden to subjects compared to bone marrow puncture to obtain bone marrow-derived mesenchymal stem cells when mesenchymal stem cells are extracted. Adipose tissue may be obtained from adipose tissues of, for example, human, mice, rats, dogs, cattle, and the like.

The hyaluronic acid is a glycosaminoglycan which is an essential element of the extracellular matrix (ECM), a kind of mucopolysaccharide consisting of an amino acid and a uronic acid, and a polymer of linear polysaccharides in which monomer N-acetylglucosamine and monomer D-glucuronic acid are continuously connected. In addition, the hyaluronic acid is a basic component of living tissue, is essential for cell morphogenesis, cell differentiation, and cell division, and is a biocompatible substance that helps restore the wound. Hyaluronic acids exhibit excellent viscoelasticity and high water absorption ability while being insoluble in an aqueous solution through an ether bond, and is maintained in a form for a certain period in the living body and is decomposed and absorbed into the body. Natural hyaluronic acids are rapidly decomposed by hyaluronidase when injected into the body. Therefore, to adjust the decomposition rate, natural hyaluronic acids are crosslinked by various methods, or their structures are modified by using a chemical substance such as benzyl alcohol to produce a hyaluronic acid derivative.

The hyaluronic acid may include hyaluronic acid itself, a salt of hyaluronic acid, or a mixture thereof. The salt of hyaluronic acid may be any salt form suitable for application into a living body. The salt of hyaluronic acid may be sodium hyaluronate, potassium hyaluronate, magnesium hyaluronate, zinc hyaluronate, cobalt hyaluronate, tetrabutylammonium hyaluronate, or a combination thereof.

The hyaluronic acid derivative may be a hyaluronic acid crosslinked product in which hyaluronic acid, hyaluronic acid salt, or a mixture thereof is crosslinked by a crosslinking agent.

The hyaluronic acid derivative solution may be a solution obtained by dissolving the hyaluronic acid derivative in a physiological saline solution, a phosphate-buffered saline solution, or a biocompatible saline solution.

The pH of the pharmaceutical composition may be from about 5 to about 8.5, from about 5.2 to about 7.5, or from about 5.5 to about 7. When the pH of the pharmaceutical composition is within the above range, the adipose-derived mesenchymal stem cells may stay inside the intervertebral disc for a long time, without affecting the survival of the adipose-derived mesenchymal stem cells.

When the pharmaceutical composition is transplanted into an intervertebral disc, cells of the surrounding human tissues migrate into the pharmaceutical composition. Even when the cells secrete the ECM and some components of the pharmaceutical composition are decomposed, the secreted ECM may sustain cell adhesion, cell survival and regeneration ability of the transplanted adipocyte-derived mesenchymal stem cells.

The volume ratio of adipose tissue-derived mesenchymal stem cells to the hyaluronic acid derivative solution may be in a range of 1:3 to 3:1, 1:2 to 2:1, 1:1.5 to 1.5:1, or about 1:1. In the case of mixing in these volume ratio ranges, the swelling ratio of the pharmaceutical composition is substantially constant even with a change in temperature or pH, so that the pharmaceutical composition is structurally stable. Therefore, the cell adhesion, cell survival, and regeneration ability of adipose tissue-derived mesenchymal stem cells may be maintained for a long time even after the pharmaceutical composition is transplanted into the intervertebral disc. In one or more embodiments, the pharmaceutical composition may be a mixture of pellets or suspension of adipose tissue-derived mesenchymal stem cells and the hyaluronic acid derivative solution.

The viscosity of the hyaluronic acid derivative solution may be in a range from about 150 Pa·s to about 900 Pa·s, from about 200 Pa·s to about 900 Pa·s, or from about 250 Pa·s to about 900 Pa·s. For example, the viscosity of the hyaluronic acid derivative solution may be in a range from about 150 Pa·s to about 900 Pa·s, from about 200 Pa·s to about 900 Pa·s, or from about 250 Pa·s to about 900 Pa·s, in a shear rate range from 200 1/s to 1400 1/s. The viscosity of the pharmaceutical composition may be in a range from about 150 Pa·s to about 900 Pa·s, from about 160 Pa·s to about 700 Pa·s, from about 170 to about 500 Pa·s, or from about 200 Pa·s to about 300 Pa·s. For example, the viscosity of the pharmaceutical composition may be in a range from about 150 Pa·s to about 900 Pa·s, from about 160 Pa·s to about 700 Pa·s, from about 170 Pa·s to about 500 Pa·s, or from about 200 Pa·s to about 300 Pa·s, in a shear rate range from 200 1/s to 1400 1/s. When the pharmaceutical composition having the above ranges of viscosity and elasticity is transplanted into the intervertebral disc, cell leakage may be prevented, and thus, abnormal development of osteophyte growth in the anterolateral intervertebral disc space may be prevented.

An amount of the hyaluronic acid derivative in the hyaluronic acid derivative solution may be in a range from about 0.7 w/v % to about 3 w/v %, from about 0.8 w/v % to about 2.5 w/v %, from about 0.8 w/v % to about 2.3 w/v %, from about 1.0 w/v % to about 2.1 w/v %, or from about 1.3 w/v % to about 2.1 w/v %. An amount of the hyaluronic acid derivative in the pharmaceutical composition may be in a range from about 0.7 w/v % to about 3 w/v %, from about 0.7 w/v % to about 2.5 w/v %, from about 0.7 w/v % to about 2.3 w/v %, from about 0.7 w/v % to about 2.1 w/v %, from about 0.7 w/v % to about 2 w/v %, from about 0.7 w/v % to about 1.8 w/v %, from about 0.8 w/v % to about 1.5 w/v %, from about 0.9 w/v % to about 1.5 w/v %, or from about 0.9 w/v % to about 1.3 w/v %. When the amount of the hyaluronic acid derivative in the pharmaceutical composition is less than 0.7 w/v %, the hyaluronic acid derivative solution may not adhere to the intervertebral disc. Accordingly, the hyaluronic acid derivative solution may not act as a cell support. When the amount of the hyaluronic acid derivative in the pharmaceutical composition is more than 3 w/v %, the viscosity and elasticity of the solution may not be uniform throughout the solution, and the solution may not be homogeneous.

A weight average molecular weight of the hyaluronic acid derivative may be in a range of about 100 Da to about 10,000 kDa, about 1,000 Da to about 6,000 kDa, about 6,000 Da to about 4,000 kDa, about 100 kDa to about 3,000 kDa, or about 300 kDa to about 3,000 kDa. Within the weight average molecular weight ranges, even when the hyaluronic acid derivative swells, the viscosity and elasticity of the hyaluronic acid derivative solution may be maintained relatively constant.

The size of the hyaluronic acid derivative may be in a range of about 100 μm to about 3,000 μm, about 100 μm to about 2,000 μm, or about 300 μm to about 1,500 μm. The hyaluronic acid derivative may be in the form of particles, microparticles or micro beads.

The degree of crosslinking of the hyaluronic acid derivative may be in a range of about 10% to about 100%, about 30% to about 90%, or about 50% to about 90%. This is to match the decomposition rate of hyaluronic acid derivatives to the initial regeneration period of cartilage.

The hyaluronic acid derivative may be a hyaluronic acid crosslinked product in which hyaluronic acid, hyaluronic acid salt, or a mixture thereof is crosslinked by a crosslinking agent. In this case, the equivalent ratio of the crosslinking agent to the repeating unit of hyaluronic acid, hyaluronic acid salt, or a mixture thereof may be in a range of about 0.01% to about 500%, about 0.1% to about 400%, about 1% to about 300%, about 10% to about 250%, or about 50% to about 150%. When the equivalent ratio of the crosslinking agent is less than 0.01%, the hyaluronic acid derivative may not be formed in the form of particles. When the equivalent ratio of the crosslinking agent is greater than 500%, the desired viscosity and elasticity may not be obtained. The viscosity and elasticity of the hyaluronic acid derivative solution may be changed by controlling the equivalent ratio of the crosslinking agent.

The crosslinking agent may be an epoxy crosslinking agent having two or more epoxy functional groups, and may be 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylpropane polyglycidyl ether, 1,2-(bis(2,3-epoxypropoxy)ethylene, pentaerythritol polyglycidyl ether, sorbitol polyglycidyl ether, or any combination thereof.

The degree of swelling of the hyaluronic acid derivative may be in a range of about 100% to about 3,000%, about 100% to about 2,000%, or about 400% to about 1200%. The degree of swelling of the hyaluronic acid derivative may be adjusted such that the hyaluronic acid derivative swells within the weight average molecular weight range and crosslinking degree range of the hyaluronic acid derivative.

Adipose tissue-derived mesenchymal stem cells may have such immunological properties as CD44⁺, CD73⁺, CD90⁺, CD105⁺, CD29⁺, CD49⁺, and human leukocyte antigen (HLA)-ABC⁺. In one or more embodiments, adipose tissue-derived mesenchymal stem cells may have such immunological properties as CD45⁻ and human leukocyte antigen (HLA)-DR⁻.

Adipose tissue-derived mesenchymal stem cells may have such immunological properties as TGF-β receptor⁺. The TGF-β receptor may be TGF-β receptor I, TGF-β receptor II, or TGF-β receptor III. Adipose tissue-derived mesenchymal stem cells, which highly express TGF-β receptor, may be able to stimulate TGF-β-1 signaling, and may further stimulate the formation of the intervertebral disc in response to autologous TGF-β-1.

Adipose tissue-derived mesenchymal stem cells may be obtained by separating adipocytes, red blood cells, cell lysates, etc. from adipose tissues obtained by liposuction or the like, by, for example, washing and filtration and then culturing them in a culture medium for stem-cell culture. Adipose tissue-derived mesenchymal stem cells may be obtained by subculturing stem cells isolated from adipose tissues for 3 to 17 passages or 3 to 14 passages. The mesenchymal stem cells derived from adipose tissue cultured in excess of 17 passages may have a tendency to slow the cell proliferation and decrease the cell viability rate.

Adipose tissue-derived mesenchymal stem cells may be in the form of a spindle.

The low back pain may be caused by degenerative changes in the intervertebral disc. Degenerative changes in the intervertebral disc may be accompanied by physiological and chemical modifications that cause pain inside the intervertebral disc. Normally, there are no nerves or vascular tissues inside the intervertebral disc. When the intervertebral disc degenerates and cracks, the sinuvertebral nerve and the blood vessels, which are distributed outside the fibrous ring, grow inward to promote degenerative changes that cause pain, and inflammatory substances such as Substance P may be actively secreted, and thus, pain caused by intradiscal disruption, which is a pathogen of pain, develops in the intervertebral disc. The low back pain may also occur with intervertebral disc herniation, bulging disc, disc protrusion, disc prolapse, disc extrusion, disc scoliosis, or spinal stenosis.

The amount of adipose tissue-derived mesenchymal stems cells may be in a range of about 1×10⁶ to about 1×10⁸ cells/ml.

The composition may be administered in a therapeutically effective amount to a subject in need. The therapeutically effective amount refers to the amount of active ingredient or pharmaceutical composition that induces a biological or medical response in a cell line, tissue system, animal or human, as contemplated by a researcher, veterinarian, physician or other clinician. The therapeutically effective amount includes an amount that induces alleviation of the symptoms of the disease or disorder being prevented or treated.

The dose of adipose tissue-derived mesenchymal stem cells may be about 5×10⁵ to about 5×10⁷ cells/subject. In one embodiment, the dose of adipose tissue-derived mesenchymal stem cells may be in a range of about 0.1×10⁵ to about 10×10⁵ cells/kg, about 0.5×10⁵ to about 10×10⁵ cells/kg, about 1×10⁵ to about 10×10⁵ cells/kg, or about 2×10⁵ to about 8×10⁵ cells/kg. When the dose is less than 5×10⁵ cells/subject or 0.1×10⁵ cells/kg based on one administration, desired intervertebral disc regeneration effects may not be obtained. When the dose is 5×10⁷ cells/subject or 10×10⁵ cells/kg based on one administration, pulmonary embolism may develop.

The hyaluronic acid derivative solution may be in the form of a hydrogel. The hydrogel refers to a gel containing water as a dispersion medium. The hydrogel may be formed when hydrosol loses its fluidity due to cooling, or when a hydrophilic polymer having a three-dimensional network structure and a microcrystalline structure expands due to containing water.

Another aspect of the present disclosure provides a method of preparing a pharmaceutical composition for preventing or treating low back pain, wherein the method includes mixing adipose tissue-derived mesenchymal stem cells and a hyaluronic acid derivative solution.

The volume ratio of adipose tissue-derived mesenchymal stem cells to the hyaluronic acid derivative solution is as described above. The volume ratio of adipose tissue-derived mesenchymal stem cells and the hyaluronic acid derivative solution may be in a range of 1:3 to 3:1, 1:2 to 2:1, 1:1.5 to 1.5:1, or about 1:1.

An amount of the hyaluronic acid derivative in the hyaluronic acid derivative solution may be in a range from about 0.7 w/v % to about 3 w/v %, from about 0.8 w/v % to about 2.5 w/v %, from about 0.8 w/v % to about 2.3 w/v %, from about 1.0 w/v % to about 2.1 w/v %, or from about 1.3 w/v % to about 2.1 w/v %. The amount of the hyaluronic acid derivative in the hyaluronic acid derivative solution and the amount of the hyaluronic acid derivative in the pharmaceutical composition are the same as described above.

A weight average molecular weight of the hyaluronic acid derivative may be in a range of about 100 Da to about 10,000 kDa, about 1,000 Da to about 6,000 kDa, about 6,000 Da to about 4,000 kDa, about 100 kDa to about 3,000 kDa, or about 300 kDa to about 3,000 kDa. The weight average molecular weight of the hyaluronic acid derivative is the same as described above.

Prior to the mixing the mesenchymal stem cell derived from adipose tissue and the hyaluronic acid derivative solution, the method may further include preparing a hyaluronic acid solution by dissolving hyaluronic acid, hyaluronic acid salt, or a mixture thereof in an aqueous alkaline solution of about 0.1 N to about 0.5 N at a concentration of about 50 mg/ml to about 250 mg/ml; and preparing a hyaluronic acid derivative solution by adding a crosslinking agent in an equivalent ratio of about 0.01% to about 500% with respect to the repeating unit of hyaluronic acid, hyaluronic acid salt, or a mixture thereof to the hyaluronic acid solution.

The hyaluronic acid, hyaluronic acid salt or a mixture thereof may be dissolved in an aqueous alkali solution at a concentration of about 50 mg/ml to about 250 mg/ml, about 75 mg/ml to about 225 mg/ml, about 75 mg/ml to about 210 mg/ml, or about 80 mg/ml to about 200 mg/ml. The hyaluronic acid, hyaluronic acid salt, or a mixture thereof may be dissolved in an aqueous alkali solution of about 0.1 N to about 0.5 N, about 0.15 N to about 0.4 N, or about 0.2 N to about 0.3 N. The alkali aqueous solution may be an aqueous alkali solution having a pH of about 9 to about 13, which may be an aqueous solution of sodium hydroxide, an aqueous solution of potassium hydroxide, or aqueous ammonia. The hyaluronic acid, the hyaluronic acid salt, or the mixture thereof is the same as described above.

The crosslinking agent may be added in an equivalent ratio of about 0.01% to about 500%, about 0.1% to about 400%, about 1% to about 300%, about 10% to about 250%, or about 50% to about 150% with respect to the repeating unit of the hyaluronic acid, the hyaluronic acid salt, or the mixture thereof. The crosslinking agent may be mixed with hyaluronic acid, hyaluronic acid salt, or a mixture thereof in a homogeneous state. The equivalent ratio of the crosslinking agent is the same as described above.

The crosslinking agent may be an epoxy-based crosslinking agent having two or more epoxy functional groups, as described above.

Adipose tissue-derived mesenchymal stem cells may have such immunological properties as CD44⁺, CD73⁺, CD90⁺, CD105⁺, CD29⁺, CD49⁺, and human leukocyte antigen (HLA)-ABC⁺. In one or more embodiments, adipose tissue-derived mesenchymal stem cells may have such immunological properties as CD45⁻ and human leukocyte antigen (HLA)-DR⁻. Adipose tissue-derived mesenchymal stem cells may have such immunological properties as TGF-β receptor⁺.

Adipose tissue-derived mesenchymal stem cells may be obtained by subculturing stem cells isolated from adipose tissues for 3 to 17 passages or 3 to 14 passages. The passage of adipose tissue-derived mesenchymal stem cells is the same as described above.

Adipose tissue-derived mesenchymal stem cells may be in the form of a spindle.

The low back pain may be caused by degenerative changes in the intervertebral disc.

The amount of adipose tissue-derived mesenchymal stems cells may be in a range of about 1×10⁶ to about 1×10⁸ cells/ml. The dose of adipose tissue-derived mesenchymal stem cells may be in a range of about 5×10⁵ to about 5×10⁷ cells/subject. In one embodiment, the dose of adipose tissue-derived mesenchymal stem cells may be in a range of about 0.1×10⁵ to about 10×10⁵ cells/kg, about 0.5×10⁵ to about 10×10⁵ cells/kg, about 1×10⁵ to about 10×10⁵ cells/kg, or about 2×10⁵ to about 8×10⁵ cells/kg.

The pharmaceutical composition for preventing or treating low back pain, the pharmaceutical composition including adipose tissue-derived mesenchymal stem cells and a hyaluronic acid derivative solution, may be provided as a cell therapeutic agent. The cell therapeutic agent is a material used for treatment, diagnosis and/or prevention through a series of actions that change the biological properties of cells, including in-vitro proliferation and screening of autologous, allogenic, or xenogenic cells to restore the functions of cells and tissues. The cell therapeutic agent is classified as a somatic cell therapeutic agent and a stem cell therapeutic agent. The pharmaceutical composition may be provided as a stem cell therapeutic agent, for example, a mesenchymal stem cell therapeutic agent.

The pharmaceutical composition may further include a pharmaceutically acceptable carrier, excipient or diluent depending on an administration method. In one or more embodiments, the pharmaceutical composition may further include saline, sterilized water, Ringer's solution, buffered saline, dextrose solution, maltodextrin solution, glycerol, ethanol, liposome, or a combination of one or more of these components, and, if needed, other conventionally used additives, such as an antioxidant or a buffer solution. According to the purpose of administration, diluents, dispersants, surfactants, binders, and lubricants may be added to the pharmaceutical composition to formulate into injectable formulations, such as aqueous solutions, suspensions, emulsions, etc., pills, capsules, granules or tablets. To allow the pharmaceutical composition to act specifically on a target organ, a target organ or tissue-specific antibody or other ligands may be used in combination with the carrier. The types of carrier, excipient or additive as described above include all conventional agents in the art, and are not limited to the carriers, excipients, or additives used in the above examples.

The pharmaceutical composition may be appropriately administered to a subject according to the purpose or necessity, depending on the conventional method, route of administration and dose used in the art. The subject may be human, mice, rats, dogs, cattle, and the like, and is not limited thereto. The subject may be any one suffering from low back pain. The low back pain may be caused by degenerative changes in the intervertebral disc. The low back pain may be caused by disc herniation, bulging disc, disc protrusion, disc prolapse, disc extrusion, disc scoliosis, or spinal stenosis. Examples of routes of administration include an oral route, a non-oral route, a intraspinal route, a subcutaneous route, or an intraperitoneal route. The appropriate dose and the appropriate frequency of administration may be determined according to a method known in the art. The amount and the administration frequency of the pharmaceutical composition to be actually administered may be appropriately determined depending on various factors, including the kind of symptoms for the prevention or treatment, the route of administration, gender, health condition, diet, the age and weight of the subject, and the severity of the disease.

Hereinafter, the present invention will be described in further detail with reference to the following examples, which should not be construed as limiting the scope of the present invention.

### Example 1: Administration of Adipose Tissue-Derived Mesenchymal Stem Cells and Hyaluronic Acid Derivative Solution and Confirmation of Reduction in Intervertebral Disc Pain

1: Preparation of Hyaluronic Acid Derivative

A hyaluronic acid derivative solution was prepared as follows. Sodium hyaluronate was dissolved at a concentration of 100 mg/ml in a 0.25 N NaOH solution. To this solution BDDE (1,4-butanedioldiglycidylether) as a crosslinking agent (butanediol diglycidyl ether was added in an equivalent ratio of 100% based on the repeating unit of the hyaluronate), and, after 36 hours of reaction at a temperature of 30° C., the reaction mixture was washed with physiological saline to remove unreacted materials therefrom. The product obtained by the washing was pulverized and the particle size was adjusted to prepare a hyaluronic acid derivative (weight average molecular weight: 300 to 3,000 kDa, and swelling degree: 400 to 1200%).

2: Preparation of Hyaluronic Acid Derivative Solution and its Physicochemical Properties

2.1: Physical Properties of Hyaluronic Acid Derivative Solution

The hyaluronic acid derivative prepared according to Example 1 was mixed with physiological saline to prepare various concentrations of the hyaluronic acid derivative, from 0.1 w/v % to 4 w/v %, at intervals of 0.1 w/v %. The physical properties of the hyaluronic acid derivative solution were measured to confirm suitable physical properties for a composition and an amount of the hyaluronic acid derivative for transplantation into the intervertebral disc. The degree of spreading of the hyaluronic acid derivative solution in a petridish, the degree of movement of the hyaluronic acid derivative solution after tilting the petridish at an angle of 45° were measured, and the rheometer analysis were used to evaluate how well the viscosity thereof was maintained with respect to the shear rate.

As a result, it was confirmed that the hyaluronic acid derivative solution having a concentration of 0.7 w/v % or more had the form of a gel. At a concentration of 0.7 w/v % to 2 w/v %, the hyaluronic acid derivative solution retained the viscosity of 150 Pa·s to 900 Pa·s. This result shows that such a concentration of the hyaluronic acid derivative solution is injectable into a target site, that is, the intervertebral disc. For example, 0.9 w/v % to 1.3 w/v % of the hyaluronic acid derivative solution had a viscosity of 200 Pa·s to 300 Pa·s and an average elasticity of about 250 Pa·s. This result shows that such a concentration of the hyaluronic acid derivative solution has optimal properties for injection into the intervertebral disc together with mesenchymal stem cells derived from adipose tissues. In the above physical properties ranges, the hyaluronic acid derivative solution is not easily decomposed in vivo and is structurally stable. Therefore, while minimizing loss of adipose tissue-derived stem cells from the intervertebral disc, even with one-time administration, the hyaluronic acid derivative solution may support adipose tissue-derived mesenchymal stem cells for about one year so that the mesenchymal stem cells stably settle. On the other hand, when the amount of the hyaluronic acid derivative in the hyaluronic acid derivative solution exceeds 3 w/v %, it was confirmed that the viscosity and elasticity of the hyaluronic acid derivative solution were not homogeneous.

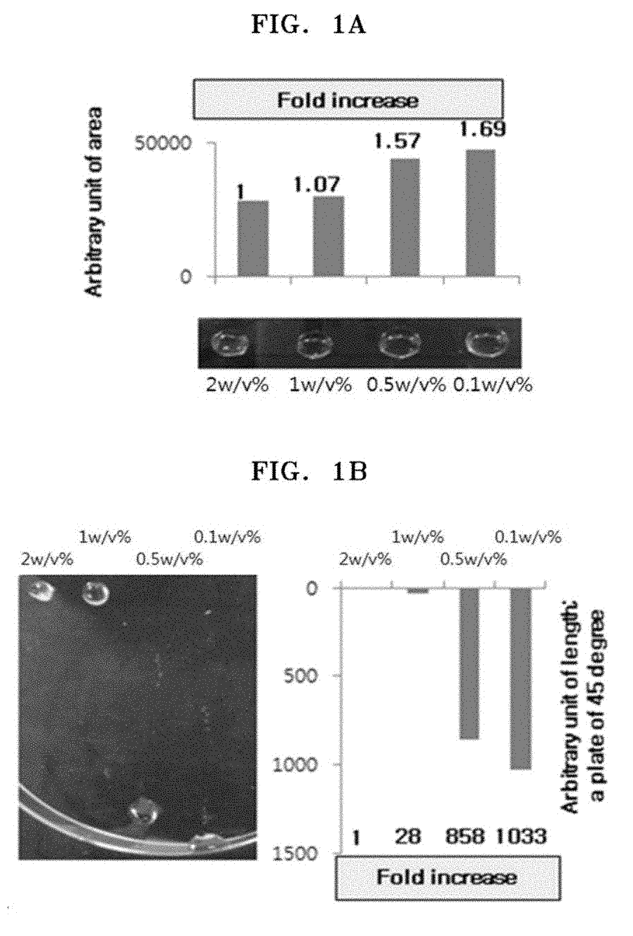

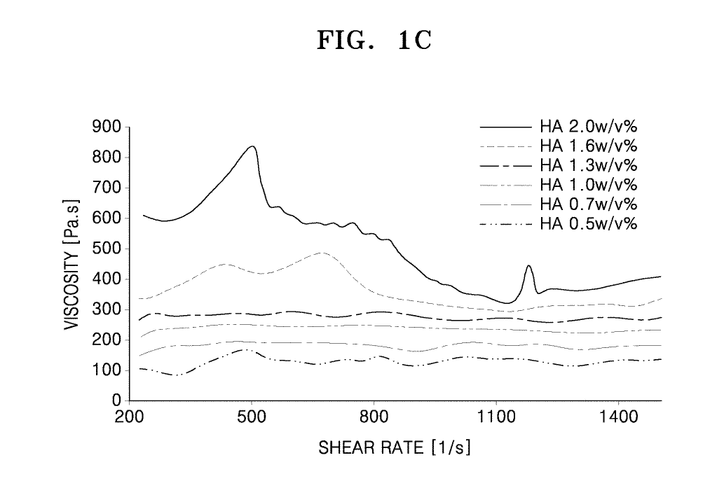

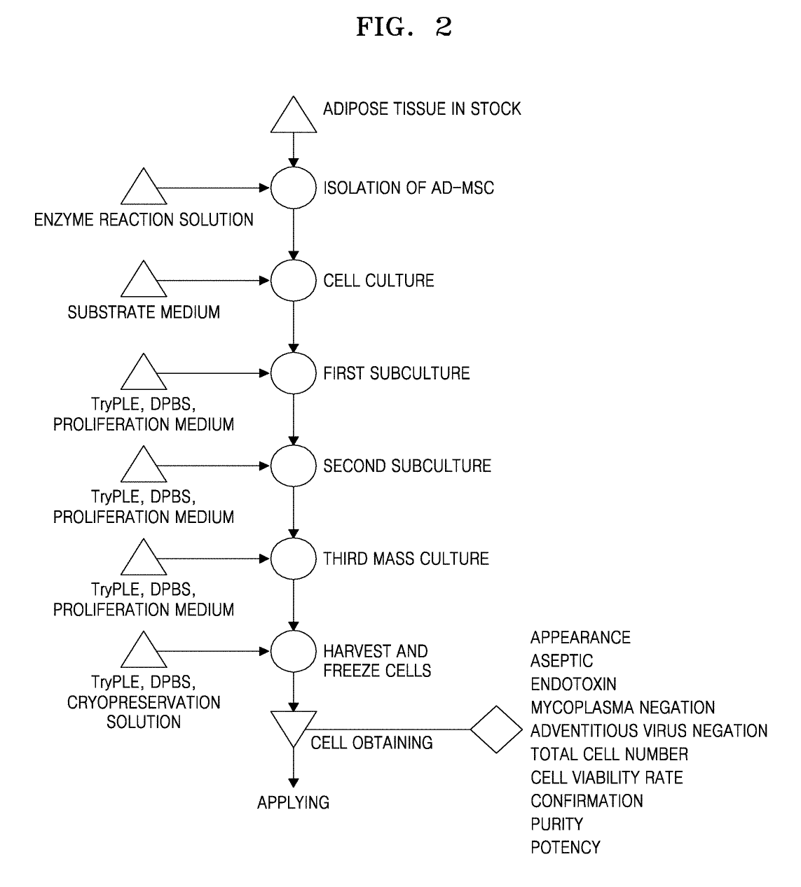

FIG. 1A to 1C shows diagrams to explain physical properties of the hyaluronic acid derivative solution at concentrations of 0.1, 0.5, 1, and 2 w/v % from among various concentrations of hyaluronic acid derivative solutions. Referring to FIGS. 1A to 1C, 2 w/v % of the hyaluronic acid derivative solution and 1 w/v % of the hyaluronic acid derivative solution hardly spread when dropped on a petridish. On the other hand, 0.1 w/v % hyaluronic acid derivative solution and 0.5 w/v % hyaluronic acid derivative solution spread on the petridish without maintaining the shape of the droplet. In addition, the degree of movement of the hyaluronic acid derivative solution after tilting the petridish at the angle of 45° was evaluated. The result shows that 2 w/v % hyaluronic acid derivative solution and 1 w/v % hyaluronic acid derivative solution did not move while adhering to the petridish.

2.2: Confirming pH of Hyaluronic Acid Derivative Solution

The pH of the hyaluronic acid derivative solution having various concentrations prepared according to Example 2.1 was measured three times according to the pH measurement method of the general test method described in the Korea Pharmacopoeia. The pH of the hyaluronic acid derivative solutions was in a range of 5.5 to 8.5.

3: Isolation of Adipose Tissue-Derived Mesenchymal Stem Cells

Ten patients with degenerative lumbar intervertebral disc herniation suffering from chronic low back pain underwent liposuction, which was carried out to collect adipose tissues from the subcutaneous fat layer of the abdominal or thigh of the patients. Cells obtained from the collected adipose tissues were proliferated and cultured, according to the following protocol.

FIG. 2 is a flowchart to explain a method of preparing mesenchymal stem cells derived from adipose tissues for transplantation into the intervertebral disc. Adipose tissue was mixed with the same volume of Dulbecco's phosphate buffered saline (DPBS), and the mixture was centrifuged and an upper oil layer was removed therefrom. The centrifuging and removing of the upper oil layer were repeated three times. Thereafter, pellets were treated with an enzyme reaction solution containing HBSS (Hank's balanced salt solution), collagenase I, trypsin, dispase, and Dnase I for 1 hour to isolate cells. Isolated cells were placed in a culture medium containing the substrate medium and cultured in an incubator supplied with 5% CO₂. The substrate medium was MEM Alpha GlutaMAX containing 10% fetal bovine serum.

When the initially cultured cells reached 70% or more of the area of the culture vessel, TrypLE was evenly distributed on the bottom, and reacted for 3 to 5 minutes in an incubator supplied with 5% CO₂, and the cells were then removed from the culture vessel. Then, the cells were cultured in a proliferation medium. Growth medium was MEM Alpha GlutaMAX containing 10 ng/ml bFGF and 10% fetal bovine serum. After subculture to the third passage, the cells were reacted with TrypLE to isolate the cells, and the cells were suspended in sterile physiological saline (normal saline).

4: Morphology and Proliferation of Isolated Cells

4.1: Morphology of Isolated Cells

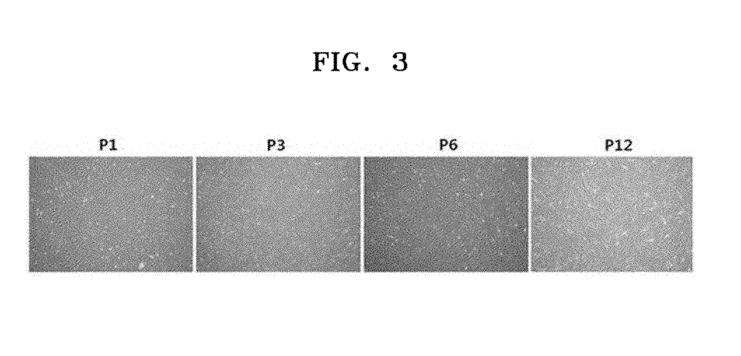

The morphological changes in the cells were observed under a microscope while the isolated cells were continuously subcultured. FIG. 3 shows images showing the morphology of isolated cells according to a passage. As a result of continuous subculture to the 12th passage, the isolated cells retained the specific shape of the spindle fibroblasts with irregular protrusions. The isolated cells showed typical mesenchymal stem cell morphology.

4.2: Analysis of Proliferative Capacity of Isolated Cells

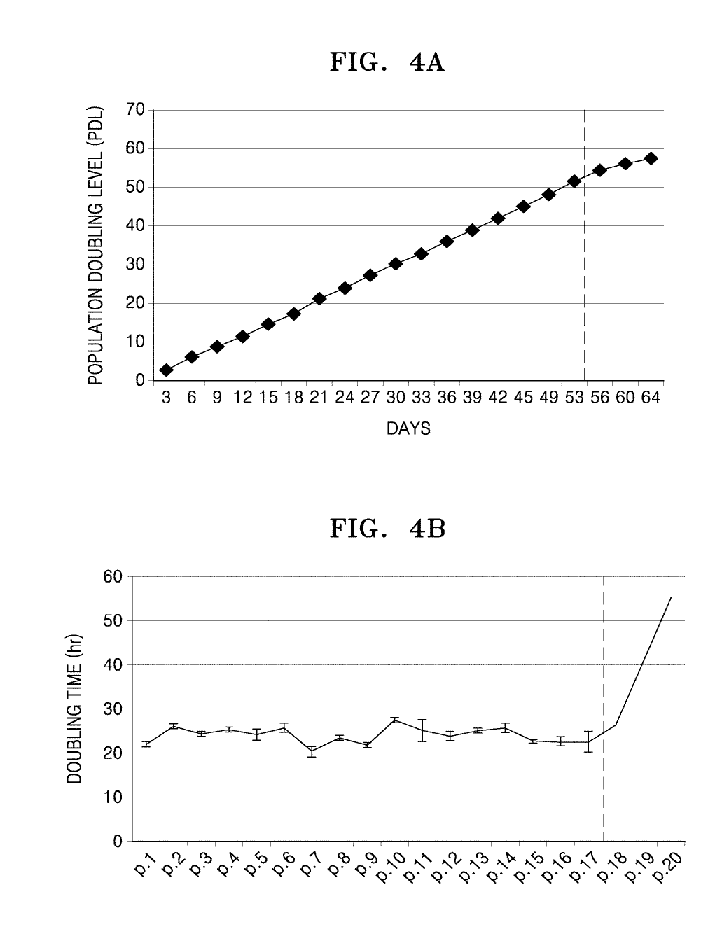

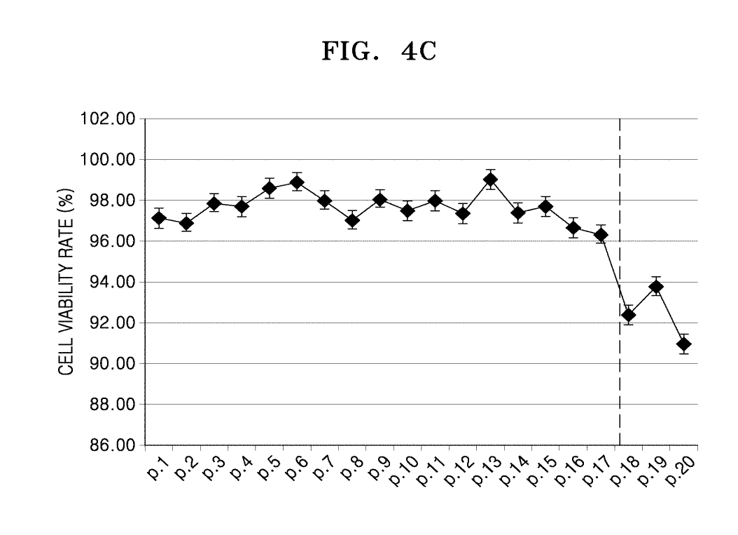

The isolated cells were continuously subcultured until the level of proliferation of the cells was slowed down. The population doubling level (PDL) of a cell group and cell division time were measured by using the trypan blue dye exclusion method at each culture step. FIGS. 4A to 4C show graphs of the PDL, the doubling time (dT), and the cell viability rate according to a passage of cell groups of isolated mesenchymal stem cells. The dT and the cell viability rate did not change to the 17th passage, and thereafter, however, cell proliferation slowed down and cell viability rate also decreased to below 95%.

5: Multipotency Analysis of Isolated Cells

Differentiation-inducing substances were added to the isolated cells to induce differentiation into adipocyte, chondrocyte, and osteoblast.

TABLE 1

| Cell lineage | Adipocyte | Chondrocyte | Osteoblast |
|---|---|---|---|
| Differentiation- | Dexamethasone | Ascorbic | Ascorbic |
| inducing | | acid | acid |
| substances | | | |
| | Isobutyl | BMP-6 | BMP-2 |
| | methylxanthine | | |
| | Indomethacin | Dexamethasone | Dexamethasone |
| | Insulin | Insulin | 1,25-dihydroxy- |
| | | | vitamine D |
| | Thiazolidinedione | Transforming | — |
| | | growth factor-β | |

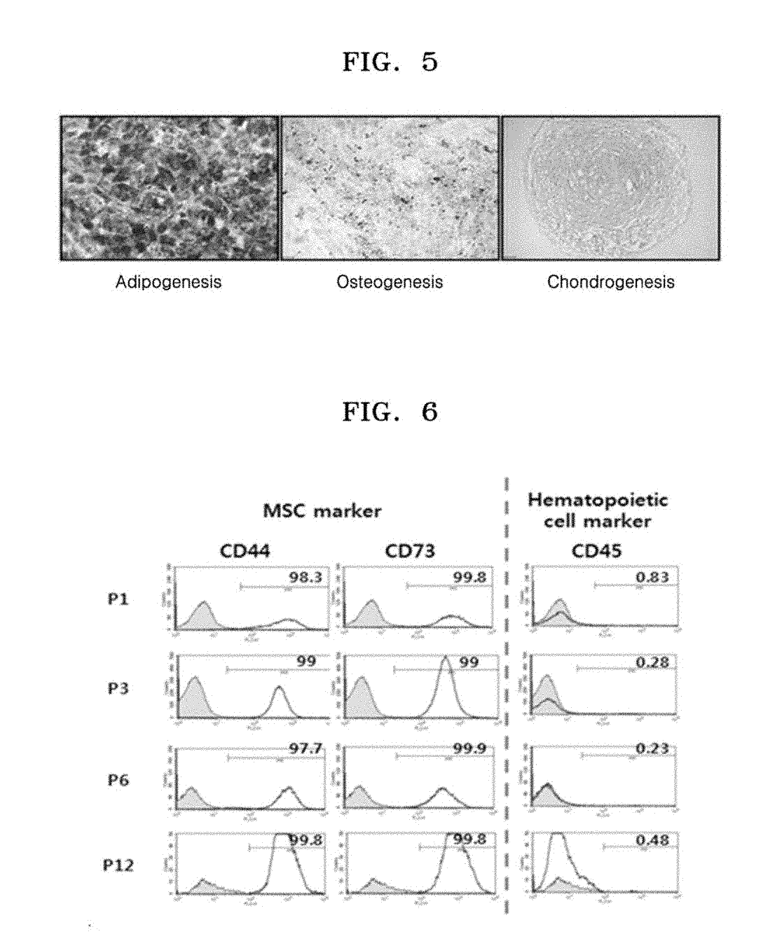

A sample of cells induced to differentiate into adipocytes were stained with Oil Red O, a sample of cells induced to differentiate into chondrocytes was stained with Alcian blue, and a sample of cells induced to differentiate into osteoblasts were stained with Alizarin Red S. FIG. 5 shows images of the isolated cells obtained by inducing differentiation into adipocytes, osteoblasts and chondrocytes by adding differentiation-inducing substances thereto. Referring to FIG. 5, it can be seen that the isolated cells have the differentiation potentials of mesenchymal stem cells, and that they were induced to differentiate into adipocytes, osteoblasts and chondrocytes.

6: Identification of Markers of Mesenchymal Stem Cells (Confirmation of Expression of CD44, CD73, CD90, CD105, CD29, CD49 and HLA-ABC)

Flow cytometry was performed to analyze the characteristics of isolated cells by using FACS accuri C6 instrument (BD Biosciences, Billerica, Mass., USA).

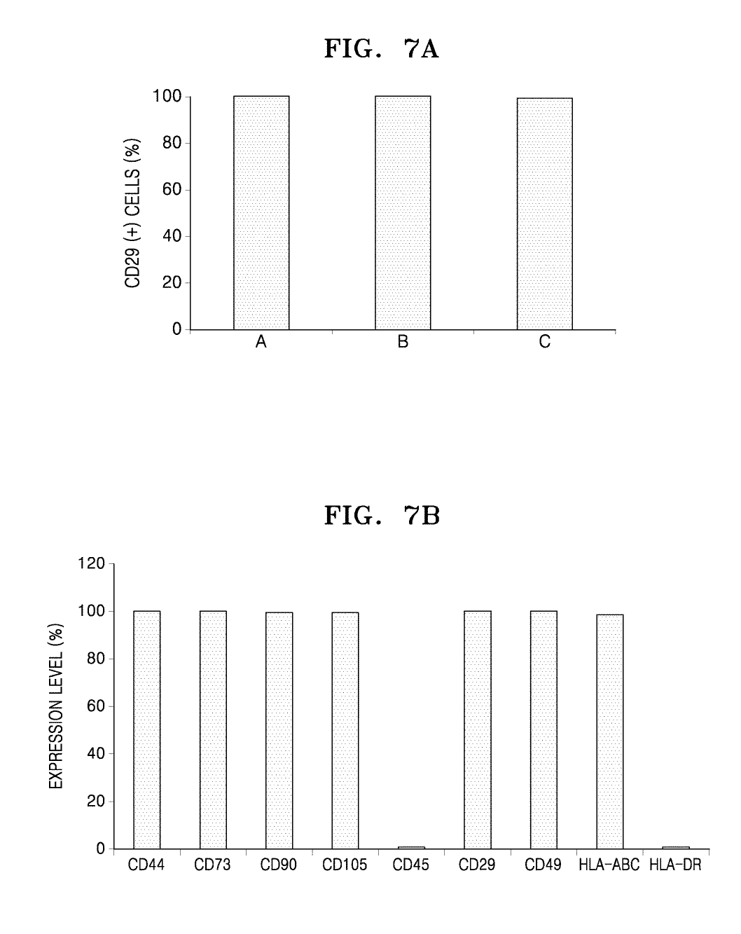

FIG. 6 is a graph showing the surface expression of the isolated cells expressing the mesenchymal stem cell marker. Referring to FIG. 6, the isolated cells expressed mesenchymal stem cell markers CD44 and CD73. Transferring cells to damaged tissues is a very important factor for a cell therapeutic agent. Among surface factors expressed on the cell surface, CD29 is a factor having a homing effect, that is, a factor that helps cells to move to the infected site or damaged tissue. The expression level of CD29 in isolated cells was confirmed. FIG. 7A shows expression results of CD29 in cells isolated from three subjects. Referring to FIG. 7A, it was confirmed that the isolated cells expressed CD29, which is a mesenchymal stem cell marker. FIG. 7B shows expression levels of CD44, CD73, CD90, CD105, CD45, CD29, CD49, and HLA-ABC, which are surface factors, in adipose tissue-derived mesenchymal stem cells. Table 2 shows the expression level of surface factors of mesenchymal stem cells. The results of Table 2 show that the expression levels of CD44, CD73, CD90, CD105, CD29, CD49, and HLA-ABC were high on the surface of isolated cells. From this result, it is seen that the isolated cells have the characteristics of mesenchymal stem cells.

TABLE 2

| | Surface factor | | | | | | | | |
|---|---|---|---|---|---|---|---|---|---|
| | CD44 | CD73 | CD90 | CD105 | CD45 | CD29 | CD49 | HLA-ABC | HLA-DR |
| Expression | 99.9 | 99.9 | 99.7 | 99.5 | 0.523 | 99.9 | 99.9 | 98.5 | 0.296 |
| level (%) | | | | | | | | | |

7: Confirming Expression of TGF-β Receptor

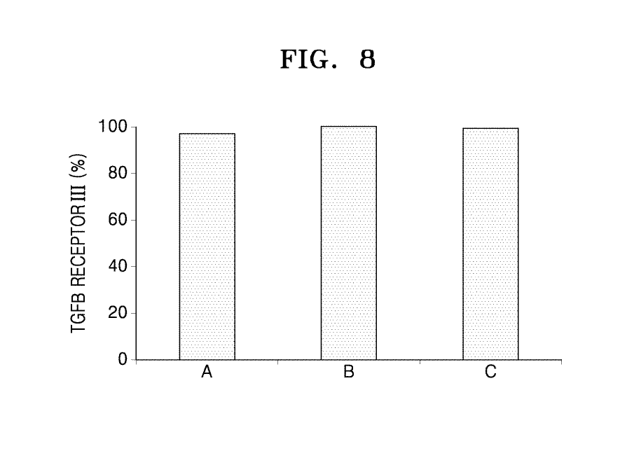

The expression level of TGF-β receptor was confirmed in isolated cells. FIG. 8 is a graph showing the expression level of TGF-β receptor III in cells isolated from three subjects. The expression level of TGF-β III in isolated cells was 98.7±1.4% on average.

8: Confirming Chromosome Stability

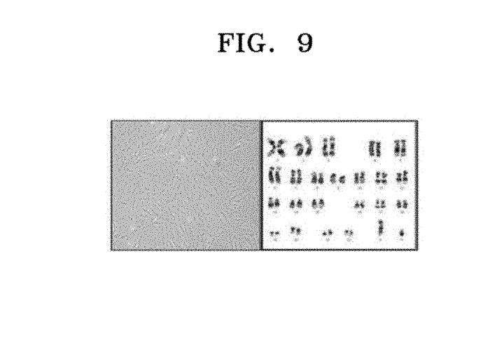

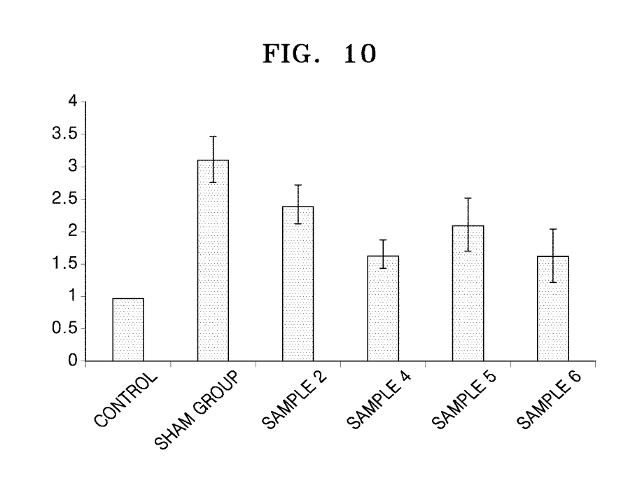

In order to continuously subculture the isolated cells, the effect of repeated cell division on the chromosome stability of the cells was confirmed. The isolated cells were subcultured to the seventh passage, and then, the proliferated cells were separated and karyotypes thereof were analyzed. FIG. 9 is an image showing the morphology and karyotype of isolated cells after subculturing to the 7th passage. Referring to FIG. 9, even when cell division is repeated, the chromosomes of the isolated cells show a normal karyotype, that is, it was confirmed that no specific mutation appears on the chromosomes.

9: Toxicity of Adipose Tissue-Derived Mesenchymal Stem Cells in Hyaluronic Acid Derivatives

CCK-8 (cell counting kit-8) analysis was performed to confirm the proliferation of adipose tissue-derived mesenchymal stem cells in the hyaluronic acid derivative. Adipose tissue-derived mesenchymal stem cells were seeded on a 96-well plate, and on the following date, 0.7, 1, 1.3, and 2 w/v % of hyaluronic acid derivative solutions were separately added to the cells. Cells cultured for 1, 3, and 7 days were washed once with phosphate buffered saline (PBS), and then 1 ml of the culture was added thereto, and 100 μl of CCK-8 (Dojindo, Tokyo, Japan) was added thereto, and the result was reacted at a temperature of 37° C. for 2 hours. 100 μl of the culture of adipose tissue-derived mesenchymal stem cells to which various concentrations of hyaluronic acid derivative solutions had been added, were placed on a 96-well plate. The absorbance of the sample was measured by using an Elisa plate reader (Power Wave X340; Bio-Tek Instruments, Inc., Winooski, Vt.) at a wavelength of 450 nm. The cell proliferation rate was calculated by using the ratio of absorbance to cell proliferation time (absorbance/day).

In addition, cytotoxicity of each adipose tissue-derived mesenchymal stem cell cultured for 3 days was verified in 2D and 3D environment by using Live/Dead analysis. The cultured adipose tissue-derived mesenchymal stem cells were washed three times with phosphate-buffered saline, and stained with 2 μM calcein AM (Invitrogen) and 4 μM ethidium homodimer (EthD-1, Invitrogen) for 5 minutes. Then, living cells (green) and dead cells (red) were observed by using a fluorescence microscope.

The cytotoxicity with respect to the hyaluronic acid derivative solution was evaluated. The evaluation results show that the level of cytotoxicity was not as high as to affect viability of the cells.

10: Administration of Adipose Tissue-Derived Mesenchymal Stem Cells and Hyaluronic Acid Derivative Solution and Recovery of Disc Water Content

A degenerative intervertebral disc (IVD) animal model was prepared according to the method described in SaKai D et al. (Biomaterial, 2003). Specifically, 28 rabbits (New Zealand white rabbits) were selected. The rabbits were 4 to 5 months old and weighed about 3 kg. Nucleus pulposus (NP) of L3/4, L4/5 and L5/6 was removed from the rabbits to prepare IVD animal models. Suctioned IVD fragments were observed under a microscope to confirm that the NP had been suctioned. The average weight of the removed NP was about 7 mg.

Among 28 rabbits in total, one rabbit, used as a control, was not induced to undergo IVD degeneration, and administered with a saline solution. A sham group consisting of three rabbits was induced to have IVD degeneration, and was administered with a saline solution. The remaining twenty-four experimental rabbits were divided into eight experimental groups, and Samples 1 to 8 of Table 3 were administered to the experimental groups, respectively. Adipose tissue-derived mesenchymal stem cells were injected at 1×10⁶ cells or 2×10⁶ cells per subject, and the amount of the hyaluronic acid derivative was adjusted to be from 0.7 w/v % to 2 w/v %.

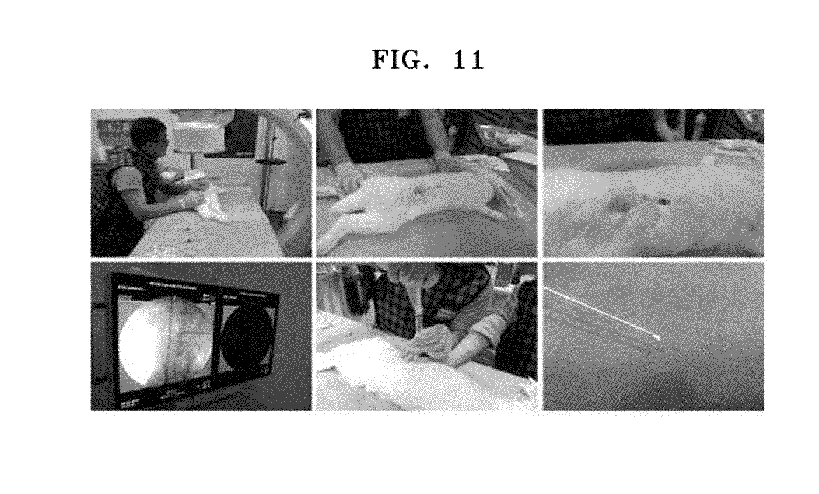

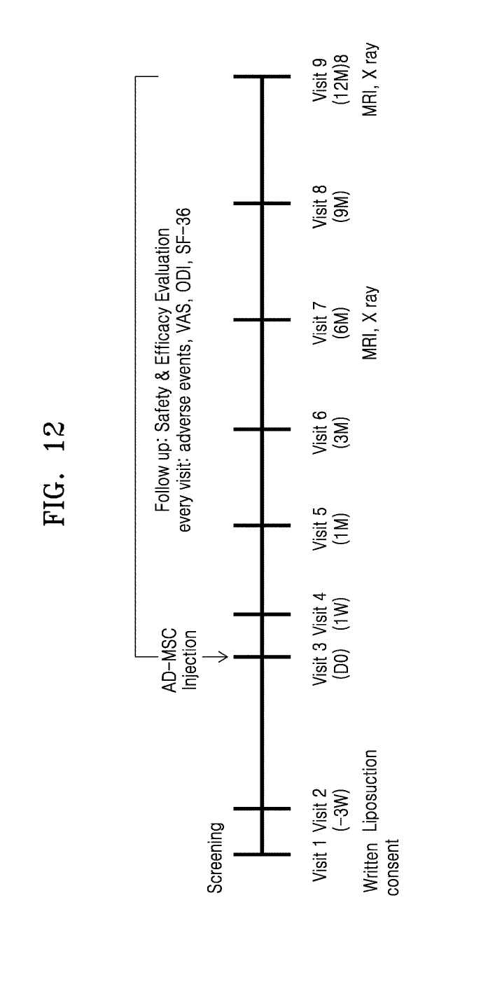

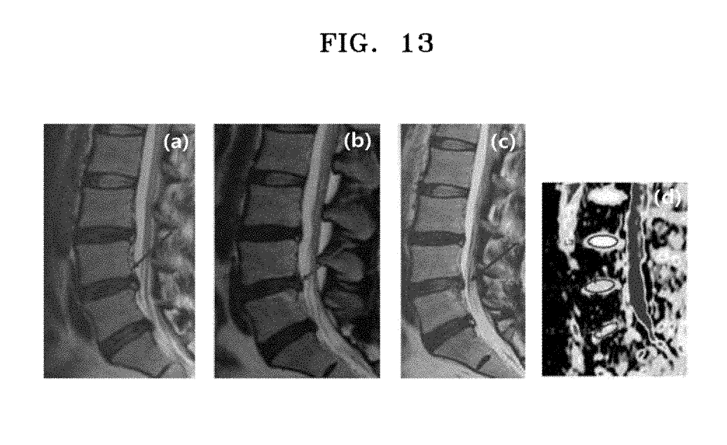

The method of administration is as follows. Adipose tissue-derived mesenchymal stem cells and/or hyaluronic acid derivative solution shown in Table 3 were administered once to the IVD of the rabbits under fluoroscopic imaging by using a 25 gauge needle (see FIG. 11).

TABLE 3

| | | Adipose tissue- | Hyaluronic |
|---|---|---|---|
| | | derived mesenchymal | acid |
| | | stem cells | derivative |
| | Sample | (1 × 10⁶ cells/25 μl) | solution |
| Control | — | — | — |
| Sham group | — | — | — |
| Experimental | Sample 1 | — | 50 μl |
| group | (Concentration: 2 | | |
| | w/v %) | | |
| | Sample2 | 25 μl | — |
| | Sample3 | 50 μl | — |
| | Sample4 | 25 μl | 25 μl |
| | (Concentration: 1 | | |
| | w/v %) | | |
| | Sample5 | 25 μl | 25 μl |
| | (Concentration: 2 | | |
| | w/v %) | | |
| | Sample6 | 25 μl | 25 μl |
| | (Concentration: 1.3 | | |
| | w/v %) | | |
| | Sample7 | 25 μl | 25 μl |
| | (Concentration: 0.9 | | |
| | w/v %) | | |
| | Sample8 | 25 μl | 25 μl |
| | (Concentration: 0.7 | | |
| | w/v %) | | |

At 3 weeks after the administration, the presence or absence of adverse reaction was observed by T2 magnetic resonance imaging (MRI), and at 6 and 12 weeks after the administration, the degree of recovery of IVD water content was confirmed by MRI. In addition, the IVD herniation grade was evaluated as Pfirrmann grade.

As a result, no adverse reaction was found after administration, and the water content in the disk was significantly restored after administration of Samples 4 to 8 as compared to Sample 1. Before administration of the pharmaceutical composition containing adipose-derived mesenchymal stem cells and the hyaluronic acid derivative solution, the MRI images of the Sham group and the experimental groups showed black IVD due to lack of water. On the other hand, MRI images of the experimental groups administered with Samples 4 to 8 showed white IVD due to the recovery of water content inside IVD. This is expected to reduce pain. Particularly, after the administration of Samples 4, 6 and 7, the water content inside the IVD was recovered to a very high degree.

FIG. 10 is a graph showing the Pfirrmann grade of the control, the sham group, and the experimental groups respectively administered with Sample 2, Sample 4, Sample 5, and Sample 6. Regarding the Pfirrmann grade, the control showed 1.00±00, the Sham group showed 3.12±0.35, the experimental group administered with Sample 2 showed 2.42±0.3, the experimental group administered with Sample 5 were 2.11±0.40, the experimental group administered with Sample 4 was 1.65±0.22, and the experimental group administered with Sample 6 was 1.63±0.41. The Pfirrmann grades of the experimental groups administered with Samples 4 or 6 were substantially low.

11: Selection of Patients with Degenerative Lumbar IVD Herniation and Transplantation of Adipose Tissue-Derived Mesenchymal Stem Cells

11.1: Selection of Patients with Degenerative Lumbar IVD Herniation

Patients with lumbar IVD herniation were selected according to the following criteria.
A) Men and women aged 19 and over, and under 70
B) No response to conservative therapy for 3 months or more performed to treat low back pain or hip pain that lasted 6 months or more
C) Oswestry disability index (ODI) of 30% or more
D) Visual analogue scale (VAS) of 4 or more
E) 3 to 4 magnetic resonance imaging (MRI) grades according to the Pfirrmann classification in between lumbar #1 and spine #1
F) Discography on a degenerative lumbar IVD confirmed by MRI causes pain consistent with as usual, and one or two IVDs cause pain
G) an agreement on stem-cell transplantation therapy
H) To diagnose IVD pain, automatic pressure control IVD discography was used. A needle (25 gauge, 6 inches) was inserted into a degenerative IVD identified by MRI, and when the tip of the needle was properly positioned at the center of the NP, contrast medium containing antibiotics was slowly injected into IVD by using an injection instrument capable of pressure measurement. The total injection volume was less than 3.5 cc and injected at the rate of 0.05 cc per second. Pressure, the location of the contrast agent, and pain response were recorded at every 0.5 cc injection of the contrast media. The contrast medium injection continued until one of the following conditions was satisfied:

First, the VAS score exceeds 6 points out of 10 points,

Second, in the case of grade 3 or greater of fibrous ring injury, the IVD internal pressure increases to 50 psi or more from open pressure; and in the case of no fibrous ring injury, the IVD internal pressure increases to 80-100 psi, and

Third, the total amount of the contrast agent injected is 3.5 cc.

The IVD pain was diagnosed based on whether a gradual increase in the IVD pressure as described above causes pain, and the caused pain was consistent with as usual.

Based on the selection criteria, ten patients with degenerative lumbar IVD herniation of the gender and age listed in Table 4 were selected.

TABLE 4

| | Gender | Age |
|---|---|---|
| Patient 1 | Female | 37 |
| Patient 2 | Female | 42 |
| Patient 3 | Female | 49 |
| Patient 4 | Male | 42 |
| Patient 5 | Male | 44 |
| Patient 6 | Male | 41 |
| Patient 7 | Female | 30 |
| Patient 8 | Male | 32 |
| Patient 9 | Male | 64 |
| Patient 10 | Male | 54 |

11.2: Administration of Adipose Tissue-Derived Mesenchymal Stem Cells and Hyaluronic Acid Derivative Solution

Ten patients were subjected to liposuction and 20 ml to 30 ml of autologous adipose tissue was collected from each patient. Three weeks after the liposuction, autologous adipose tissue-derived mesenchymal stem cells and hyaluronic acid derivative solutions (performed at a volume ratio of 1:3 to 3:1 determined according to clinical judgment, see Table 5 below) were injected once to IVD of the patients by using a spinal needle.

The spinal needle insertion site was 10 cm to 14 cm from the midline depending on the target patient, and the tip of the spinal needle was inserted into the center of the disc using a C-shaped projector. In order to minimize the loss of stem cells, the needle was removed after keeping the needle immobilized for about 5 minutes.

TABLE 5

| | Adipose tissue- | |
|---|---|---|
| | derived | Hyaluronic acid |
| | mesenchymal | derivative |
| | stem cells | solution |
| Sample 1 | 500 μl | 1.5 ml |
| (Concentration: 1 w/v %) | (2 × 10⁷ cells) | (Concentration: 1.3 w/v %) |
| Sample 2 | 1 ml | 1 ml |
| (Concentration: 1 w/v %) | (4 × 10⁷ cells) | (Concentration: 2 w/v %) |

At all time points, including about 1 week, about 1 month, about 3 months, about 6 months, about 9 months, and about 12 months after the administration of autologous adipose tissue-derived mesenchymal stem cells and hyaluronic acid derivative solution, no adverse reactions were observed with adipose tissue-derived mesenchymal stem cells and the hyaluronic acid derivative solution.

12: Administration of Adipose Tissue-Derived Mesenchymal Stem Cells and Hyaluronic Acid Derivative Solution and Confirmation of Reduction in IVD Pain Through MRI Image

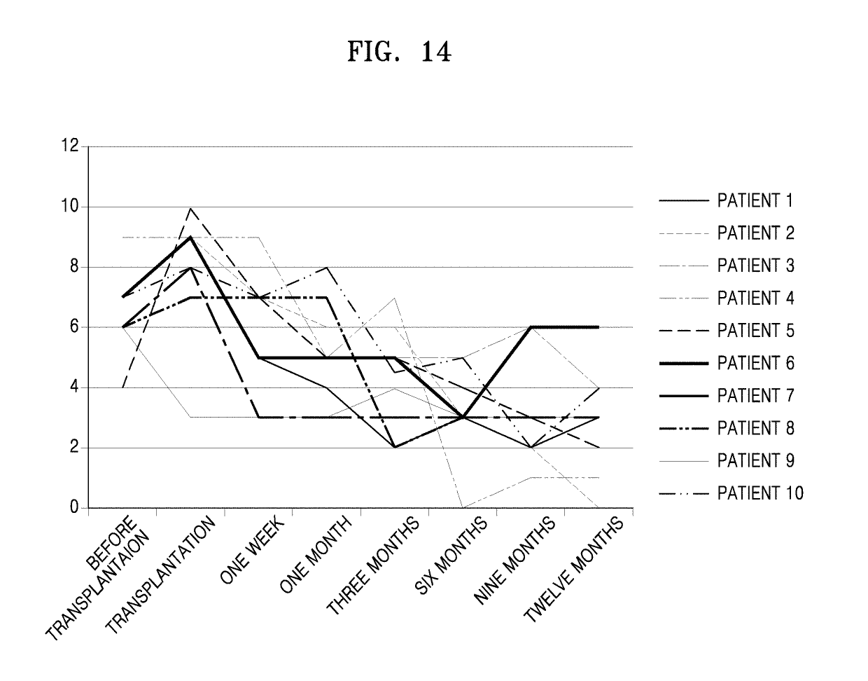

The IVD were observed by MRI before transplantation and 1 month and 6 months after the transplantation. The results of observation before and after transplantation of Patient 1 are shown in FIG. 13 as a representative example. The MRI technique is obtainable by using a T2 map, a diffusion weighted image, and an apparent diffusion coefficient (ADC) map. The results of MRI of Patient 1 administered with Sample 1 show that the water content in the IVD increased. This means that the degenerative disc has been reproduced. Similar results were obtained from Patients 2 to 10.

After the transplantation of autologous adipose tissue-derived mesenchymal stem cells, the ECM may be restored to recover the water content compared to before the transplantation of autologous adipose tissue-derived mesenchymal stem cells, and the degenerative IVD is regenerated and thus the pain due to the degenerative IVD is reduced.

13: Administration of Adipose Tissue-Derived Mesenchymal Stem Cells and Hyaluronic Acid Derivative Solution, and Confirmation of IVD Pain Reduction Based on Visual Analogue Scale (VAS)

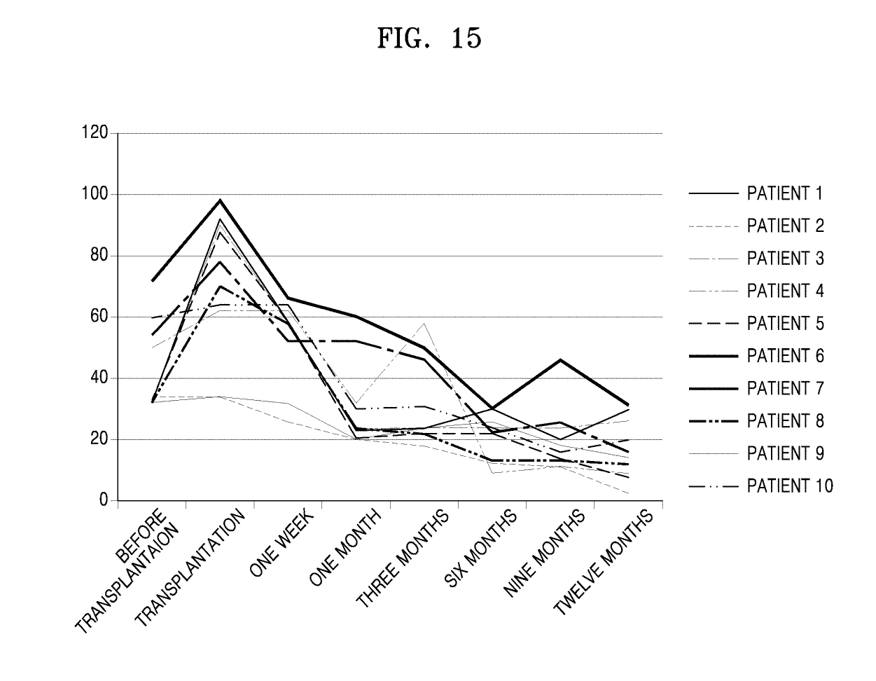

The VAS of IVD was evaluated before transplantation, and about 1 week, about 1 month, about 3 months, about 6 months, about 9 months, and about 12 months after the transplantation. The VAS scores of Patients 1 to 10 before and after the transplantation are shown in Table 6 and FIG. 14. The VAS was evaluated with a line scaled in such a way that no pain was present at one end of the line, the most severe pain imaginable was present at the end of the other, and the degree of pain felt by a patient was written on the line.

When the autologous adipose tissue-derived mesenchymal stem cells and the hyaluronic acid derivative solution were administered, the pain experienced by each of the 10 patients gradually decreased over time. When the VAS is grade 4 or higher, it was considered that there is moderate pain. About 12 months after the transplantation, 7 out of 10 patients showed VAS grade of 0 to 3.

TABLE 6

| | Patient | Patient | Patient | Patient | Patient | Patient | Patient | Patient | Patient | Patient |
|---|---|---|---|---|---|---|---|---|---|---|
| | 1 | 2 | 3 | 4 | 5 | 6 | 7 | 8 | 9 | 10 |
| Before | 9 | 7 | 6 | 7 | 4 | 7 | 6 | 6 | 6 | 7 |
| transplantation | | | | | | | | | | |
| Transplantation | 9 | 9 | 8 | 9 | 10 | 9 | 8 | 7 | 3 | 8 |
| 1 week | 5 | 7 | 7 | 9 | 7 | 5 | 3 | 7 | 3 | 7 |
| 1 month | 4 | 6 | 5 | 5 | 5 | 5 | 3 | 7 | 3 | 8 |
| 3 months | 2 | 6 | 5 | 7 | 5 | 5 | 3 | 2 | 4 | 4.5 |
| 6 months | 3 | 3 | 5 | 0 | 4 | 3 | 3 | 3 | 3 | 5 |
| 9 months | 2 | 2 | 6 | 1 | 3 | 6 | 3 | 3 | 3 | 2 |
| 12 months | 3 | 0 | 4 | 1 | 2 | 6 | 3 | 3 | 3 | 4 |

14: Administration of Adipose Tissue-Derived Mesenchymal Stem Cells and Hyaluronic Acid Derivative Solution and Confirmation of IVD Pain Reduction Based on Oswestry Disability Index (ODI)

The ODI of IVD was evaluated before transplantation, and about 1 week, about 1 month, about 3 months, about 6 months, about 9 months, and about 12 months after the transplantation. The ODI scores of Patients 1 to 10 before and after the transplantation are shown in Table 7 and FIG. 15. The ODI was evaluated with respect to items such as pain level, personal hygiene (washing, dressing), lifting, walking, sitting, standing, sleeping, social life (activities for amity and hobbies), and travel. 0-20% indicates minimal disability, 21-40% indicates moderate disability, 41-60% indicates severe disability, 61-80% indicates crippling back pain, and 81 to 100% indicates bed-bound patients who are lying alone or exaggerated patients.

When the autologous adipose tissue-derived mesenchymal stem cells and the hyaluronic acid derivative solution were administered, the degree of disability experienced by each of the 10 patients gradually decreased over time. About 12 months after transplantation, 7 out of 10 patients showed the ODI of 0 to 20%, that is, a mild level of disability.

TABLE 7

| | Patient | Patient | Patient | Patient | Patient | Patient | Patient | Patient | Patient | Patient |
|---|---|---|---|---|---|---|---|---|---|---|
| | 1 | 2 | 3 | 4 | 5 | 6 | 7 | 8 | 9 | 10 |
| Before | 32 | 34 | 30 | 50 | 32 | 72 | 54 | 32 | 32 | 60 |
| transplantation | | | | | | | | | | |
| Transplantation | 92 | 34 | 90 | 62 | 88 | 98 | 78 | 70 | 34 | 64 |
| 1 week | 58 | 26 | 58 | 62 | 58 | 66 | 52 | 58 | 32 | 64 |
| 1 month | 23 | 20 | 20 | 32 | 20 | 60 | 52 | 24 | 20 | 30 |
| 3 months | 24 | 18 | 22 | 58 | 22 | 50 | 46 | 22 | 24 | 31 |
| 6 months | 30 | 12 | 24 | 9 | 22 | 30 | 22 | 13 | 26 | 24 |
| 9 months | 20 | 11 | 24 | 11 | 14 | 46 | 26 | 13 | 18 | 16 |
| 12 months | 30 | 2.2 | 26 | 8.9 | 8 | 31.1 | 15.6 | 12 | 14 | 20 |

The pharmaceutical composition including adipose tissue-derived stem cells and the hyaluronic acid derivative solution according to embodiments of the present disclosure is capable of controlling the viscosity of the hyaluronic acid derivative solution according to the reaction conditions of the hyaluronic acid and the hyaluronic acid derivative. Mesenchymal stem cells, which have a remarkable chondrocyte differentiation ability, are mixed with the hyaluronic acid derivative solution, and the mixture is transplanted into the intervertebral disc. By doing so, diseases and low back pain caused by degenerative changes in the intervertebral disc may be effectively prevented or treated.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A method of preventing or treating low back pain in a subject, the method comprising administering to a subject in need thereof a therapeutically effective amount of a composition including adipose tissue-derived mesenchymal stem cells and a hyaluronic acid derivative solution.

2. The method of claim 1, wherein an amount of a hyaluronic acid derivative in the hyaluronic acid derivative solution is in a range of about 0.7 w/v % to about 3 w/v %.

3. The method of claim 1, wherein an amount of the hyaluronic acid derivative in the composition is in a range of about 0.7 w/v % to about 2.5 w/v %.

4. The method of claim 1, wherein a viscosity of the hyaluronic acid derivative solution is in a range of about 150 Pa·s to about 900 Pa·s.

5. The method of claim 1, wherein a volume ratio of adipose tissue-derived mesenchymal stem cells to the hyaluronic acid derivative solution is in a range of 1:3 to 3:1.

6. The method of claim 2, wherein the hyaluronic acid derivative has a weight average molecular weight of 100 Da to 10,000 kDa.

7. The method of claim 2, wherein the hyaluronic acid derivative is a hyaluronic acid crosslinked product in which hyaluronic acid, hyaluronic acid salt, or a mixture thereof is crosslinked by a crosslinking agent, and an equivalent ratio of the crosslinking agent to a repeating unit of the hyaluronic acid, the hyaluronic acid salt, or the mixture thereof is in a range of about 0.01% to about 500%,

8. The method of claim 7, wherein the crosslinking agent is 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylpropane polyglycidyl ether, 1,2-(bis(2,3-epoxypropoxy)ethylene, pentaerythritol polyglycidyl ether, sorbitol polyglycidyl ether, or any combination thereof.

9. The method of claim 1, wherein adipose tissue-derived mesenchymal stem cells are selected from CD44⁺, CD73⁺, CD90⁺, D105⁺, CD29⁺, CD49⁺, and HLA (human leukocyte antigen)-ABC⁺,
CD45⁻ and HLA (human leukocyte antigen)-DR⁻, and
TGF-β receptor⁺.

10. The method of claim 1, wherein the low back pain develops due to degenerative changes in the intervertebral disc.

11. The method of claim 1, wherein an amount of adipose tissue-derived mesenchymal stem cells is in a range of about 1×10⁶ cells/ml, to about 1×10⁸ cells/ml.

12. The method of claim 1, wherein a dose of adipose tissue-derived mesenchymal stem cells is in a range of about 0.1×10⁵ cells/kg to about 10×10⁵ cells/kg.

13. A method of preparing a composition, the method comprising mixing adipose tissue-induced mesenchymal stem cells with a hyaluronic acid derivative solution.

14. The method of claim 13, wherein an amount of a hyaluronic acid derivative in the hyaluronic acid derivative solution is in a range of about 0.7 w/v % to about 3 w/v %.

15. The method of claim 13, wherein an amount of the hyaluronic acid derivative in the composition is in a range of about 0.7 w/v % to about 2.5 w/v %.

16. The method of claim 13, wherein a volume ratio of adipose tissue-derived mesenchymal stem cells to the hyaluronic acid derivative solution is in a range of 1:3 to 3:1.

17. The method of claim 14, wherein the hyaluronic acid derivative has a weight average molecular weight of 100 Da to 10,000 kDa.

18. The method of claim 13, wherein
prior to the mixing of adipose tissue-derived mesenchymal stem cells with the hyaluronic acid derivative solution,
preparing a hyaluronic acid solution by dissolving hyaluronic acid, hyaluronic acid salt, or a mixture thereof in an aqueous alkaline solution of about 0.1 N to about 0.5 N at a concentration of about 50 mg/ml to about 250 mg/ml; and
preparing a hyaluronic acid derivative solution by adding, to the hyaluronic acid solution, a crosslinking agent in an equivalent ratio of about 0.01% to about 500% with respect to a repeating unit of the hyaluronic acid, the hyaluronic acid salt, or the mixture thereof.

19. The method of claim 18, wherein the crosslinking agent is 1,4-butanediol diglycidyl ether (BDDE), ethylene glycol diglycidyl ether (EGDGE), 1,6-hexanediol diglycidyl ether, propylene glycol diglycidyl ether, polypropylene glycol diglycidyl ether, polytetramethylene glycol diglycidyl ether, neopentyl glycol diglycidyl ether, polyglycerol polyglycidyl ether, diglycerol polyglycidyl ether, glycerol polyglycidyl ether, tri-methylpropane polyglycidyl ether, 1,2-(bis(2,3-epoxypropoxy)ethylene, pentaerythritol polyglycidyl ether, sorbitol polyglycidyl ether, or any combination thereof.

20. The method of claim 13, wherein an amount of adipose tissue-derived mesenchymal stem cells is in a range of about 1×10⁶ cells/ml to about 1×10⁸ cells/ml.
